(19) United States
(12) Patent Application Publication
(10) Pub. No.: US 2024/0277426 A1
(43) Pub. Date: Aug. 22, 2024

(54) ROBOTIC SURGICAL SYSTEM HAVING REPLACEABLE SUTURING TOOL MODULE

(71) Applicant: BOSTON SCIENTIFIC SCIMED, INC., MAPLE GROVE, MN (US)

(72) Inventors: John Mims, Austin, TX (US); Luis Amador, JR., Pflugerville, TX (US); Jason N. Mehta, Austin, TX (US); Thomas Neudeck, Austin, TX (US); Michael Fries, Kingsland, TX (US); Vladimir Mitelberg, Austin, TX (US); Andrew MacLeod, Cambridge (GB); Douglas Ivan Jennings, Royston (GB)

(73) Assignee: BOSTON SCIENTIFIC SCIMED, INC., MAPLE GROVE, MN (US)

(21) Appl. No.: 18/582,284

(22) Filed: Feb. 20, 2024

Related U.S. Application Data
(60) Provisional application No. 63/447,256, filed on Feb. 21, 2023.

(51) Int. Cl. A61B 34/30; A61B 17/00; A61B 17/04

(52) U.S. Cl. CPC A61B 34/30; A61B 17/0469; A61B 2017/00477; A61B 2034/301

(57) ABSTRACT

Mounting systems configured to operably couple a tool module with respect to a component of a medical instrument. In some aspects, the component to which the tool module is operably coupled is an elongate member that is suitable for passage through a natural orifice of the patient. The tool module may include a replaceable end cap having a needle holder adapted to pass a needle through tissue, and an actuator adapted to control movement of the needle holder arm. A suture needle with suture may be removably coupled to the needle holder. The instrument may be operably associated with a robotic system for robotic control of the component of the instrument with which the tool module is operably coupled, and/or the tool module.

### CROSS-REFERENCE TO RELATED APPLICATIONS

This application claims the benefit of priority under 35 U.S.C. § 119 to U.S. Provisional Application No. 63/447,256, filed Feb. 21, 2023, the entire disclosure of which is hereby incorporated by reference herein for all purposes.

### BACKGROUND OF THE INVENTION

### 1. Field of the Invention

The present disclosure relates to systems, such as treatment systems, and, more particularly, such as robotic treatment systems, for performing a procedure with respect to a mammalian body, such as treating a mammalian body. More particularly, the present disclosure relates to tools configured for coupling with a component of the treatment system, and methods of coupling (and uncoupling) the tools to other components of the treatment system. Various aspects of the present disclosure are particularly applicable to robotic treatment systems and facilitate use of tools with features in common with tools for non-robotic systems.

### 2. State of the Art

The condition of obesity means an individual has too much body fat and also that an individual's weight is higher than what is considered to be healthy for their height. Biology plays a big role in why some people become obese, but not getting enough exercise, eating more food than the body can use, and drinking too much alcohol also contributes to people becoming obese. Obesity is a major health threat because excess weight puts more stress on every part of the body and puts people at risk of several health problems, such as diabetes, heart disease, and stroke.

For some people, lifestyle changes like maintaining a healthy diet and exercising regularly can help them drop body fat and stop being obese. For others though, it can be extremely difficult to lose body fat and consistently maintain weight loss. Medications for losing weight are available on the market, but some can have serious side effects and may not actually be effective. For obese individuals who cannot lower their amount of body fat through lifestyle changes or medications, various surgical options have become available.

Gastric bypass surgery was the first commonly practiced procedure performed to make the stomach smaller. The procedure involves stapling portions of the stomach wall together and then relocating a small part of the small intestine to the newly formed stomach pouch. By reducing the size of the stomach, the stomach holds less food, the individual obtains a sensation of fullness quicker, fewer calories are eaten, fewer calories are absorbed, and weight loss results. However, there are downsides to the procedure. The procedure is an open surgical procedure which has its own risks, including the potential for complications and infection, and can have an extensive post-surgical recovery period. The procedure is also relatively complicated requiring a reconfiguration of the small intestines. Also, over time the staples can release allowing the stomach to re-enlarge, rendering the procedure less effective.

Another procedure is the ‘gastric banding’ procedure, primarily with the LAP-BAND® system, in which an inflatable band is inserted through the abdomen and about the stomach in a laparoscopic procedure. The band is wrapped around the upper part of the stomach to form a stoma, or ring. Attached to the ring is a thin tube leading to an access port that is implanted under the skin. A balloon attached to the band contacts the stomach and can be inflated (or deflated) with saline via the access port using a needle. Adding saline tightens the stoma about the stomach to cause an earlier sensation of satiety. If the band is too tight, saline can be withdrawn. An advantage of the gastric banding is that it can be performed in a minimally invasive manner with small laparoscopic incisions into the abdomen with consequent reduced recovery time, and that no reconfiguration of the small intestines is required. Nevertheless, the procedure still requires incisions, infection can result, and the recovery can be uncomfortable. In addition, the patient is left with a permanent port just under their skin which can be undesirable to some.

These types of procedures, when all goes well, can be effective, but as stated come with the risks associated with open or laparoscopic surgery, and for that reason they are only prescribed in cases of extreme obesity.

Incisionless fully endoscopic methods of reducing the capacity of the stomach have been developed to surgically treat obesity. Broadly, such methods endoluminally approximate tissue at a portion of the stomach, including at least a portion of the greater curvature of the stomach. The method includes making a pattern of endoscopic stitches in which a significant portion of the stomach is closed off. The resulting stomach reduction procedure can provide a seventy to seventy-five percent reduction in available stomach volume. Because the procedure is incisionless, it is safer to patients and offers an easier recovery.

While the procedure is shown to be an effective method to reduce the capacity of the stomach, create an earlier sensation of satiety, and effect weight loss in an obese patient, there remain obstacles to its application. Bariatric procedures have conventionally been practiced by bariatric surgeons who approach stomach reduction from outside the stomach and are not as familiar with surgery on gastrointestinal structures when viewed from inside the gastrointestinal tract. Meanwhile, gastrointestinal surgeons who are more familiar operating on the stomach from the interior do not have the familiarity with bariatric procedures and as a consequence have a reduced comfort level with such surgeries.

Robotic systems have been used in various surgical procedures, but are not widely used in surgery in the gastrointestinal space. It is a difficult space in which to operate as the space changes significantly in shape throughout the procedure, proper orientation of the robotic tools to the reconfigured surgical space is difficult to maintain, and the required tools are different than those currently available for the surgical robotic market.

### SUMMARY OF THE INVENTION

Systems are provided for securely coupling a tool module to a component of a medical instrument, such as a surgical instrument, for use in a patient's body. For the sake of convenience and without intent to limit, reference may be made herein to surgical tools or surgical tool modules, although the tools or tool modules may be used for procedures with respect to a patient which may be considered non-surgical. Moreover, it will be appreciated that reference may be made interchangeably to tools or tool modules without intent to limit. Furthermore, reference may be made herein, interchangeably and without intent to limit, to surgical systems, treatment systems, robotic systems, etc., with which the medical instrument and tool modules are to be used. In some aspects, the instrument is a part of a system such as a robotic system, such as a robotically controlled system (e.g., controlled with automation rather than manually by a human operator). It will be appreciated that reference may be made herein specifically to a robotic instrument and/or a robotic system and coupling of a tool module to a component thereof. However, it will be appreciated that various principles of the present disclosure are applicable to other systems, such as manually-operated systems, such as manually-operated surgical systems, such as manually-operated endoscopic systems.

In some aspects, the component to which the tool module is coupled is an elongate member, such as a flexible elongate member, insertable into the patient's body. For instance, the component may have a distal end that is suitable for passage through a natural orifice of the patient, such as into the gastrointestinal tract, and particularly into the stomach. The tool module may be coupled to the distal end of the component. The tool module may include an end cap. In some aspects, the end cap is removably positioned on and/or removably operably coupled with a component of the system (e.g., an elongate member) so that the tool module may be used during a procedure, and removed for cleaning, disposal, modification, etc., and/or replacement with a different tool module. In some aspects, the tool is a suturing tool. In some aspects, the suturing tool has an end cap having a needle holder adapted to pass a needle through tissue. In some aspects, an actuator is operatively coupled with the needle holder and adapted to actuate the needle holder, such as to cause the needle holder to move. A suture needle with suture may be removably coupled to the needle holder.

In accordance with various aspects of the present disclosure, before a surgical procedure, the tool module can be operably associated with (e.g., securely mounted to) a component of a robotic instrument, such as to the distal end of such component. Then, during the procedure, the robotic system actively operates the tool module to carry out the surgical procedure. After the surgical procedure has been completed, the module may be removed from the robotic instrument. The removed tool module can be cleaned and sterilized for subsequent use, or discarded. Another (e.g., same or different) tool module may be operably associated with the component of the robotic instrument.

In accordance with various aspects of the present disclosure, various mounting systems are provided for securely mounting the tool module to a component of a system such as a robotic system. The mounting systems include, but are not limited to, mechanical connections (e.g., interengaging structures), magnetic and/or electromagnetic connections, and adhesive connections. Each of the mounting systems similarly permits the tool module to be released from a component of a system (e.g., from a distal end of the component) such as a robotic system, without damage to the system. The mounting system also secures the tool module to the component so that the component and the robotic system may effectively operate the tool module to perform a desired procedure.

It will be appreciated that although embodiments of the present disclosure may be described with specific reference to surgical devices and systems and procedures for treating the gastrointestinal system, it should be appreciated that such medical devices and methods may be used to treat tissues of the abdominal cavity, digestive system, urinary tract, reproductive tract, respiratory system, cardiovascular system, circulatory system, and the like.

### DETAILED DESCRIPTION OF THE DRAWINGS

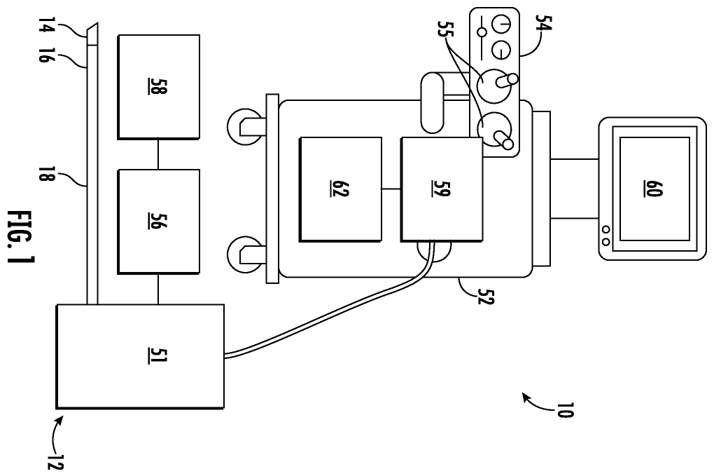

FIG. 1 is a general diagram of a robotic system.

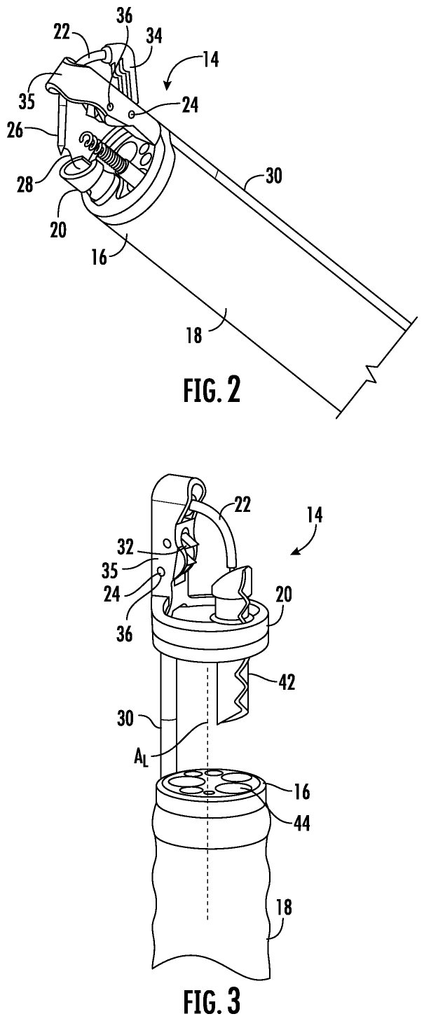

FIG. 2 is a perspective distal end view of an example of an embodiment of a tool module attached to a component of a robotic instrument of a robotic system.

FIG. 3 is a perspective assembly view of the tool module of FIG. 2 and the distal end of the robotic instrument.

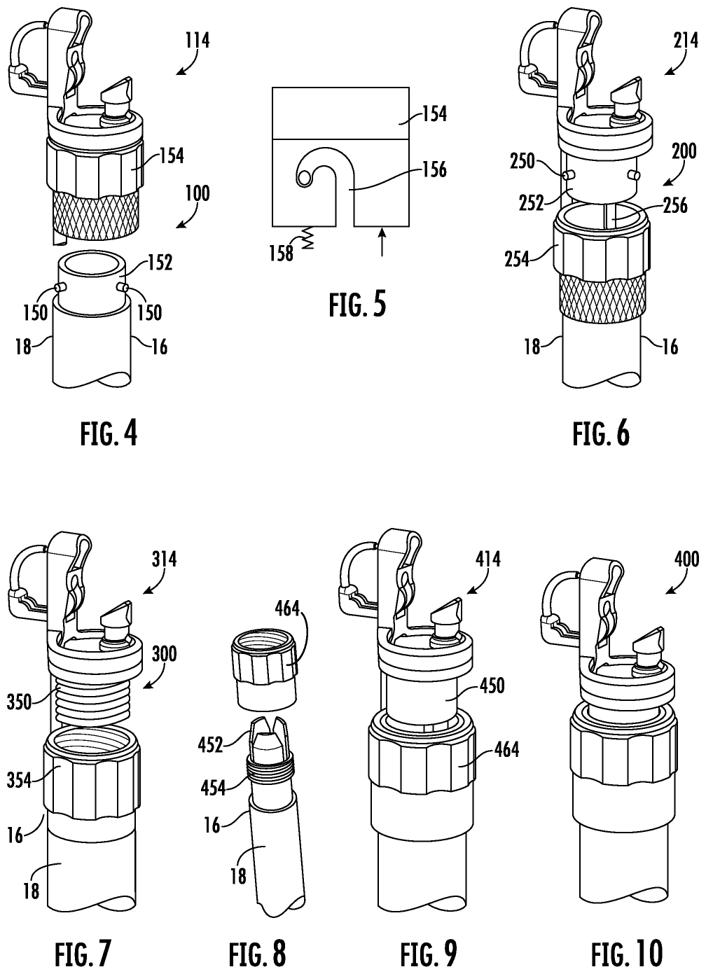

FIG. 4 is an assembly view of a first example of an embodiment of a mounting system for the tool module to the distal end of the robotic instrument.

FIG. 5 is a schematic illustration of a locking system of the mounting system of FIG. 4.

FIG. 6 is an assembly view of a second example of an embodiment of a mounting system for the tool module to the distal end of the robotic instrument.

FIG. 7 is an assembly view of a third example of an embodiment of a mounting system for the tool module to the distal end of the robotic instrument.

FIGS. 8, 9, and 10 illustrate assembly of a fourth example of an embodiment of a mounting system for the tool module to the distal end of the robotic instrument.

FIG. 11 is a section view of a fifth example of an embodiment of a mounting system for the tool module to the distal end of the robotic instrument.

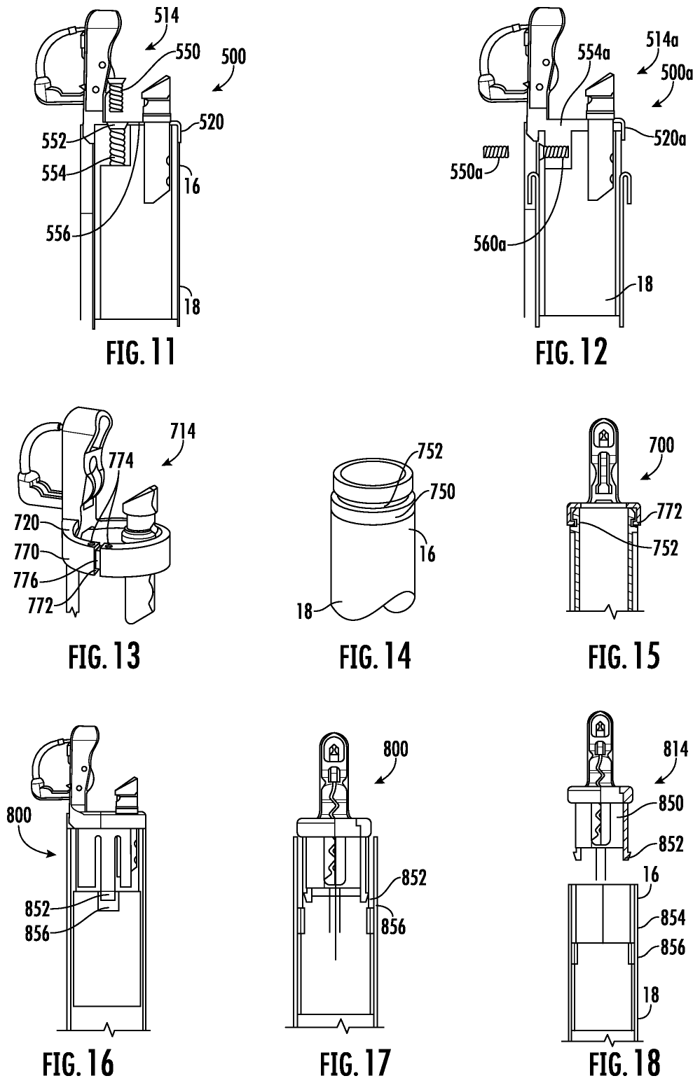

FIG. 12 is a section view of a sixth example of an embodiment of a mounting system for the tool module to the distal end of the robotic instrument.

FIGS. 13, 14, and 15 illustrate a seventh example of an embodiment of a mounting system for the tool module to the distal end of the robotic instrument.

FIGS. 16, 17, and 18 illustrate an eighth example of an embodiment of a mounting system for the tool module to the distal end of the robotic instrument.

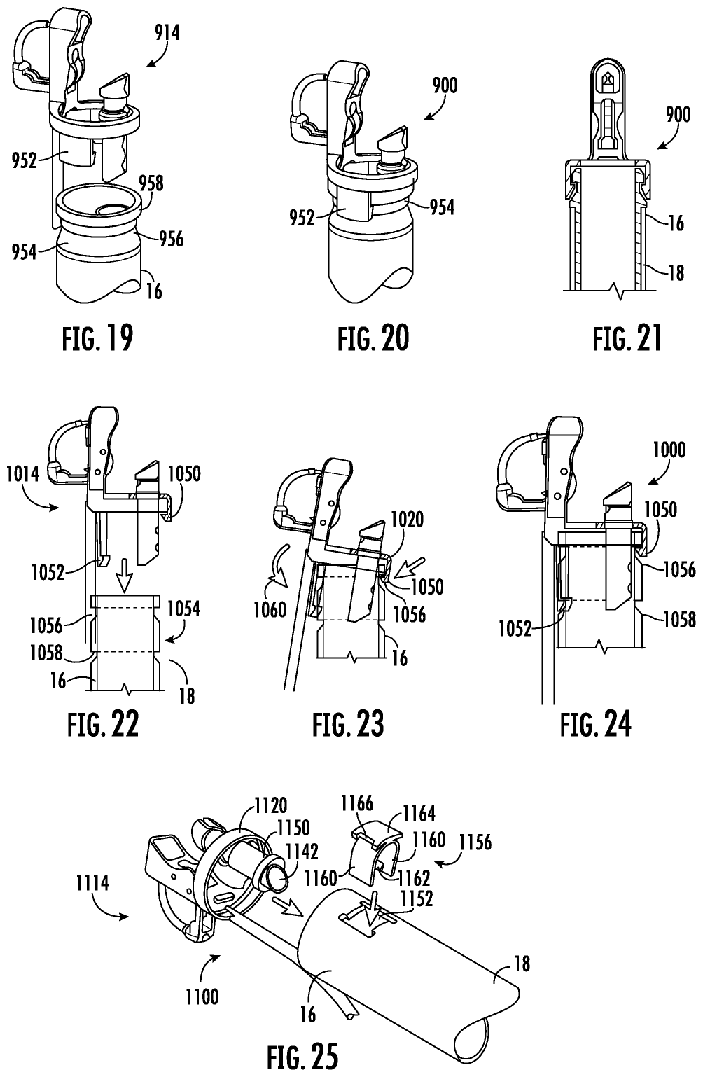

FIGS. 19, 20, and 21 illustrate a ninth example of an embodiment of a mounting system for the tool module to the distal end of the robotic instrument.

FIGS. 22, 23, and 24 illustrate a tenth example of an embodiment of a mounting system for the tool module to the distal end of the robotic instrument.

FIG. 25 is an assembly view of an eleventh example of an embodiment of a mounting system for the tool module to the distal end of the robotic instrument.

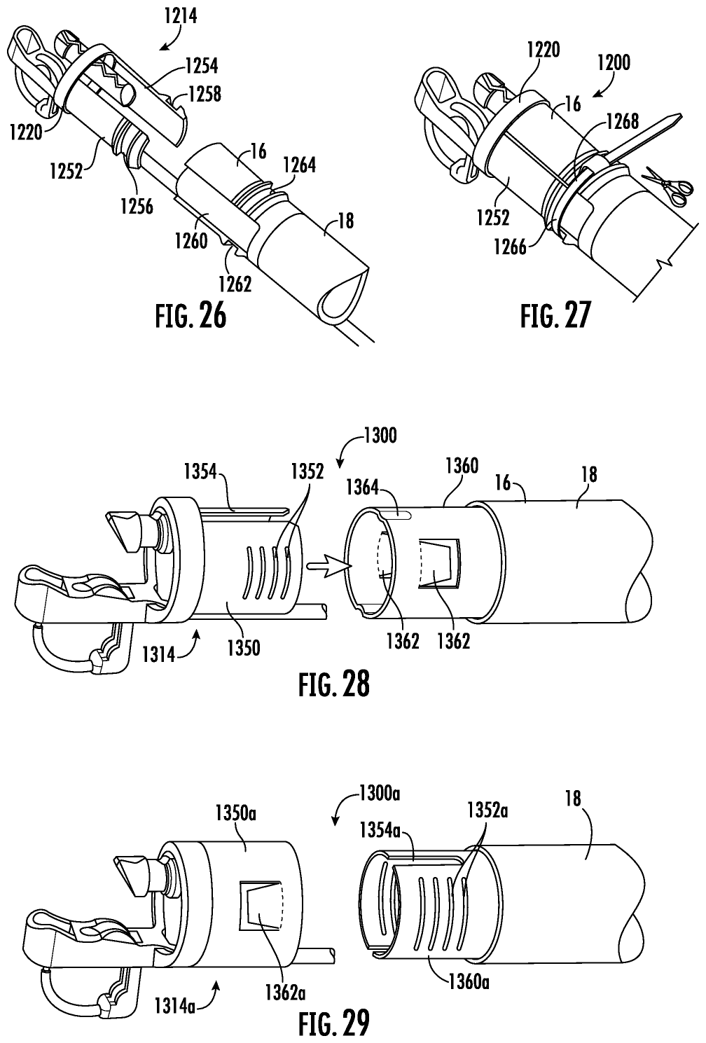

FIGS. 26 and 27 illustrate a twelfth example of an embodiment of a mounting system for the tool module to the distal end of the robotic instrument.

FIG. 28 illustrates a thirteenth example of an embodiment of a mounting system for the tool module to the distal end of the robotic instrument.

FIG. 29 illustrates a fourteenth example of an embodiment of a mounting system for the tool module to the distal end of the robotic instrument.

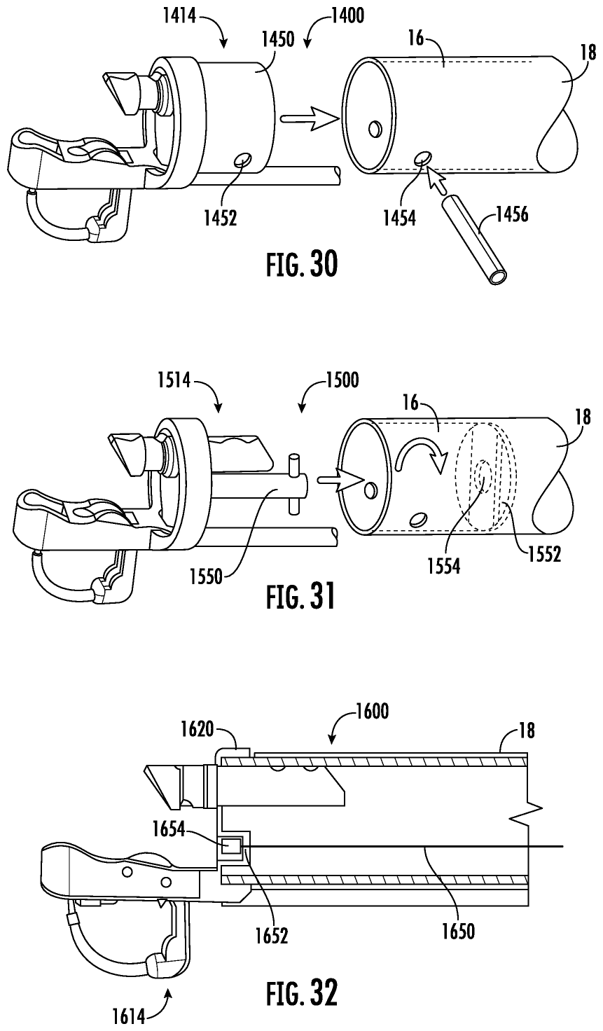

FIG. 30 illustrates a fifteenth example of an embodiment of a mounting system for the tool module to the distal end of the robotic instrument.

FIG. 31 illustrates a sixteenth example of an embodiment of a mounting system for the tool module to the distal end of the robotic instrument.

FIG. 32 illustrates a seventeenth example of an embodiment of a mounting system for the tool module to the distal end of the robotic instrument.

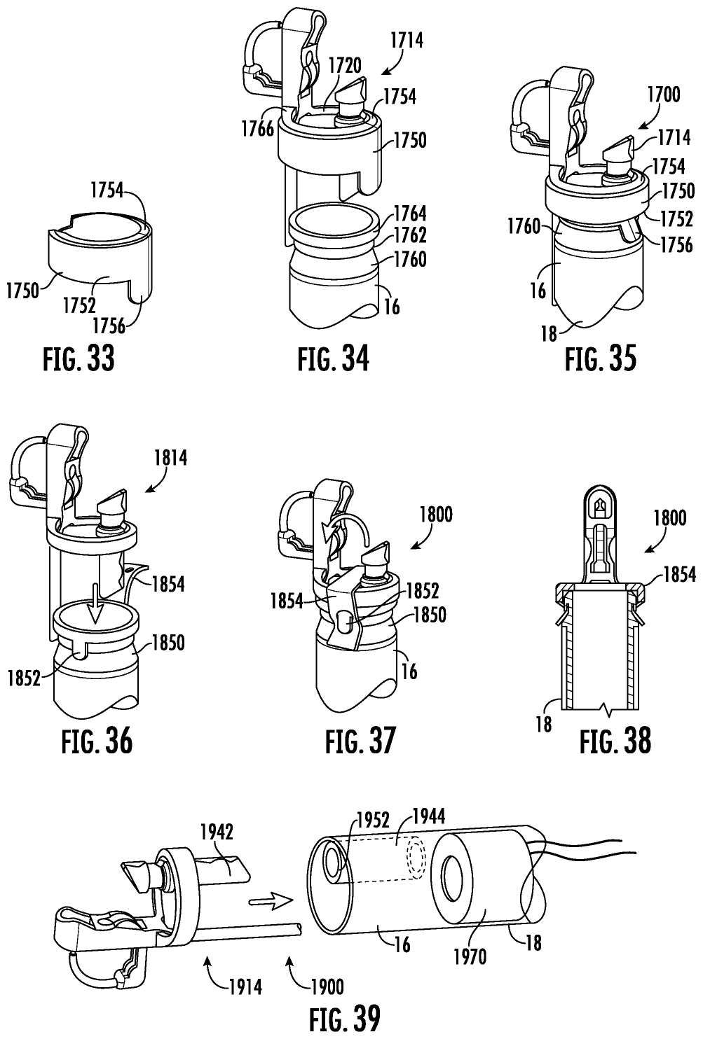

FIGS. 33, 34, and 35 illustrate an eighteenth example of an embodiment of a mounting system for the tool module to the distal end of the robotic instrument.

FIGS. 36, 37, and 38 illustrate a nineteenth example of an embodiment of a mounting system for the tool module to the distal end of the robotic instrument.

FIG. 39 illustrates a twentieth example of an embodiment of a mounting system for the tool module to the distal end of the robotic instrument.

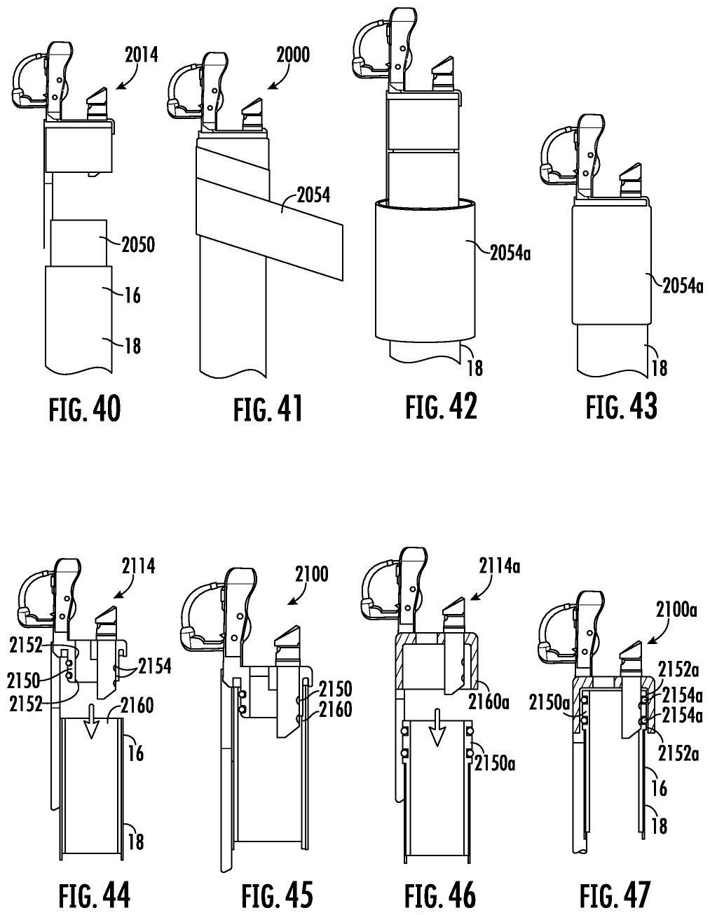

FIGS. 40 and 41 illustrate a twenty-first example of an embodiment of a mounting system for the tool module to the distal end of the robotic instrument.

FIGS. 42 and 43 illustrate an alternative twenty-first example of an embodiment of a mounting system for the tool module to the distal end of the robotic instrument.

FIGS. 44 and 45 illustrate a twenty-second example of an embodiment of a mounting system for the tool module to the distal end of the robotic instrument.

FIGS. 46 and 47 illustrate an alternate twenty-second example of an embodiment of a mounting system for the tool module to the distal end of the robotic instrument.

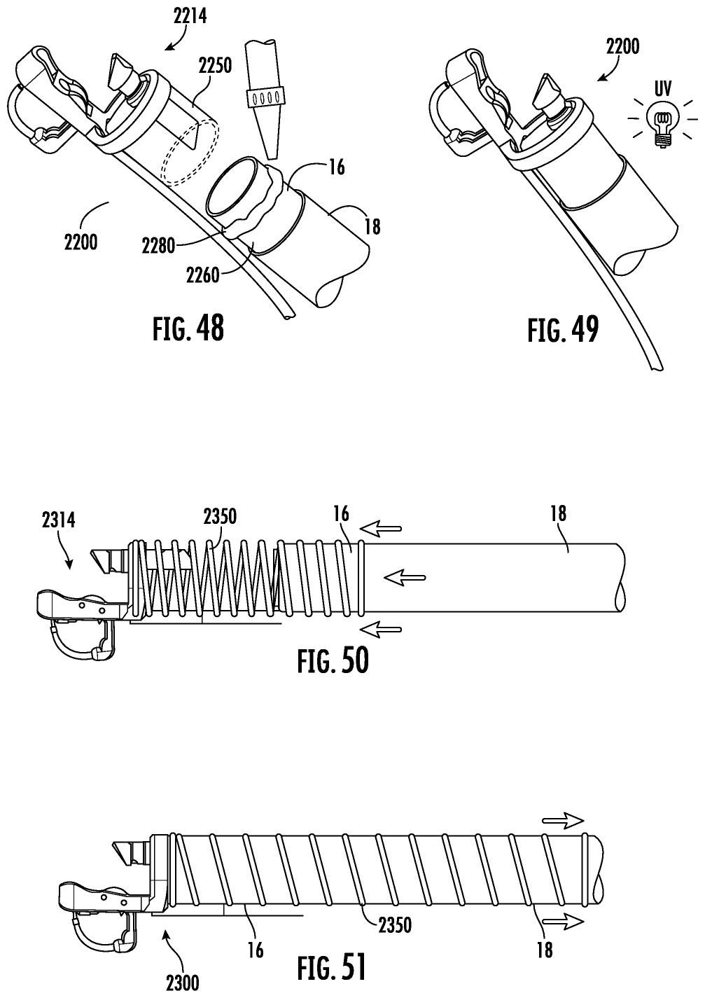

FIGS. 48 and 49 illustrate a twenty-third example of an embodiment of a mounting system for the tool module to the distal end of the robotic instrument.

FIGS. 50 and 51 illustrate a twenty-fourth example of an embodiment of a mounting system for the tool module to the distal end of the robotic instrument.

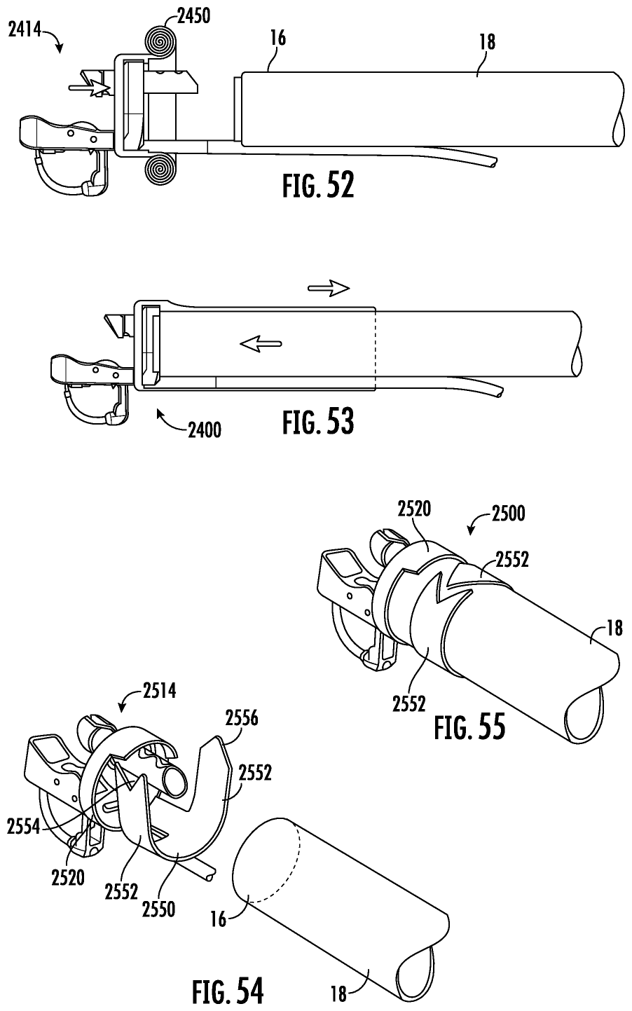

FIGS. 52 and 53 illustrate a twenty-fifth example of an embodiment of a mounting system for the tool module to the distal end of the robotic instrument.

FIGS. 54 and 55 illustrate a twenty-sixth example of an embodiment of a mounting system for the tool module to the distal end of the robotic instrument.

### DESCRIPTION OF THE INVENTION

Turning now to FIG. 1, in one example of an embodiment of a system 10, such as a robotic system 10, for performing a medical procedure, the system 10 includes an instrument 12, such as a robotic instrument 12, with a tool module 14 operably associated therewith. In some aspects, the instrument 12 is a medical instrument configured to perform a procedure, such as a surgical procedure, with respect to a patient. In some aspects, the instrument 12 is configured for insertion into a patient. In some aspects, the instrument 12 is an endoluminal instrument 12. In some aspects, the instrument 12 includes an elongate member, such as an insertion tube 18, advanceable into a natural orifice of a patient. It will be appreciated that reference is made herein to an insertion tube 18 for the sake of convenience, and without intent to limit the instrument 10 or operable association of the tool module 14 with the system 10 and/or instrument 12 by such reference to specifically an insertion tube. In some aspects, the insertion tube 18 is capable of being navigated through tortuous body passages within a patient, such as initially through a natural orifice into such body passage. In some aspects, the insertion tube 18 includes a visualization device (e.g., camera, fiber optics, etc.) and may be similar to an insertion tube of a medical scope such as an endoscope. In some aspects, the insertion tube 18 is flexible and/or shapeable. In some aspects, the system 10 and/or instrument 12 is aware of the shape of the insertion tube 18 and/or the location of the insertion tube 18 in space. In some aspects, the tool module 14 is operably associated with a distal end 16 of the instrument 12. In some aspects, the tool module 14 is configured to be readily removed (e.g., decoupled) from the instrument 12. The tool module 14 may be removed for cleaning, disposal, modification, etc., and/or replacement with a same or different tool module 14.

In some aspects, the robotic system 10, and, in some cases, the robotic instrument 12 in particular, includes a mechanized system 51 to control the movement of at least a component of the instrument 12, such as the insertion tube 18, and/or devices coupled thereto (such as a component of the tool module 14, such as a needle holder arm 22, and/or components associated therewith, as illustrated in FIG. 2). The mechanized system 51 may control the insertion tube 18 via mechanical actuators, such as, without limitation, push-pull actuators and/or rotatable actuators and/or a gear drive mechanism like a worm gear. Alternatively or additionally, the system 10 may include a control system 52 operably associated with the mechanized system 51 to provide input to mechanized system 51 based on any of a variety inputs to or from the system 10. In some aspects, a human interface 54 is operably associated with the robotic system 10, such as to convert human manual input to movement of the insertion tube 18. The interface 54 can include, without limitation, joysticks 55, trackballs, a keyboard, buttons, knobs, haptic gloves, and/or any other suitable interface to permit input from an operator. The robotic system 10, and, in some cases, the robotic instrument 12 in particular, may include first sensors 56, including, without limitation, load cells and/or strain gauges coupled to the actuators operably associated with the mechanized system 51. The sensor 56 may be configured to monitor forces applied by the mechanized system and actuators. The first sensors 56 may be located within the insertion tube 18 or coupled via mechanical, optical, and/or electrical components to sensors external to the insertion tube 18. Signals from the sensor 56 are usable by the mechanized system 51, and/or the robotic system 10, to control one or more components of the system 10 and/or the instrument 12. For instance, signals from the sensor 56 may be used to control a component of the instrument 12, such as the insertion tube 18, such as to control operation of the instrument 12 and/or insertion tube 18 and/or tool module 14. The robotic system 10, and, in some cases, the robotic instrument 12 in particular, also may include a light source and camera 58, and a visual display 60 to display images from the camera 58, optionally augmented by input from the first and second sensors and/or patient data. Other sensors 59 may be provided to sense and identify the patient environment and optionally components of the instrument 12, such as the tool module 14 (e.g., a needle and/or suture operably associated therewith). The robotic system 10 also includes a processor 62, which may include a microprocessor, that runs, for example, the software of the robotic system 10, a memory for storing software, and an interface to access patient data, and which integrates the inputs from the subsystems together to facilitate operation of the insertion tube 18 and tool module 14 to perform a surgical procedure.

While various tool modules 14 are within the scope of the present system, in one example of an embodiment, the tool module 14 is adapted to advance a suture needle through tissue. Turning to FIGS. 2 and 3, one example of an embodiment of a suturing tool module 14 includes an end cap 20 adapted for placement at and connection to the distal end 16 of the insertion tube 18. The illustrated end cap 20 includes a needle holder arm 22 operably associated with the end cap 20. In some aspects, the needle holder arm 22 is rotatable to move a needle 26 provided with a length of suture 28 through patient tissue. In some aspects, the needle holder arm 22 is rotatably mounted to move in an arc about a rotation axis 24 which extends transverse to the long axis AL of the insertion tube 18. The needle 26 is connected to the needle holder arm 22 and is adapted to pierce tissue. In some aspects, the needle 26 is removably coupled with the needle holder arm 22. In the illustrated example of an embodiment of a mechanical coupling, a flexible transmission member 30, a gear train 32 coupled to a distal end portion of the transmission member 30, and a connecting member 34 pivotably coupled to a mounting bracket 35 at pivot pin 36 and extending to the needle holder arm 22 are arranged to rotate the needle holder arm 22. It will be appreciated that other configurations of transmission members operably associated with the needle holder arm 22 to actuate the needle holder arm 22 (e.g., by to transmitting movement to the needle holder arm 22) are within the scope and spirit of the present disclosure, the present disclosure not being limited in this regard. The modules optionally may include a tubular member 42 adapted to be inserted into a tubular opening 44, such as a working channel, defined in the insertion tube 18 to prevent rotation of the module 14 relative to the insertion tube 18. In some aspects, the tubular member 42 is positioned off-axis from the axial center of the module. In some aspects, the tubular member 42 may be in the form of a split spring having an outward force that is adapted to enhance engagement between the tool module 14 and the insertion tube 18. In one embodiment, the suturing system is operable substantially as described in U.S. Pat. No. 9,867,610, which is hereby incorporated herein in its entirety. In alternative embodiments, the needle holder arm 22 and mechanical coupling may be configured to move the needle holder arm 22 and/or the needle 26 in a different manner, such as in a linear motion or in a circular motion.

As indicated above, the tool module 14 is operably associated with the instrument 12, such as mounted to the distal end 16 of the insertion tube 18, such as to be removable therefrom and/or optionally to allow replacement thereof (with a same or different type of tool module). The tool module 14 can be operably associated with the instrument 12 via a mounting system. The mounting system is shaped and configured and adapted to facilitate operable association of a tool module 14 with an instrument 12, the tool module 14 being formed separately from the instrument 12. Such operable association is selected, configured, adapted, etc., to transmit movements from the instrument 12, such as movements of the insertion tube 18, to the tool module 14, such as to operate the tool module 14, such as to perform a procedure with respect to a patient. In some aspects, the mounting system is shaped and configured and adapted to facilitate removal of the tool module 14 from the instrument 12, such as to clean, dispose of, modify, etc., the tool module 14, and/or to replace the tool module 14 with another tool module. In accordance with various principles of the present disclosure, the mounting system may include mounts which can be generally categorized as mechanical connections (e.g., interengaging structures), magnetic and/or electromagnetic connections, friction fit connections, and/or temporary adhesive connections with respect to the insertion tube 18. It will be appreciated that various connections may include multiple types of connections and be cross-categorized. Each of the mounts may be configured to permit the tool module 14 to be released from the robotic system 10 (e.g., from a component thereof, such as instrument 12, and, more particularly, such as from the insertion tube 18) without damage to the robotic system 10.

It will further be appreciated that a transmission member 30 which may be operably associated with the operable portions of the tool module 14 (e.g., the needle holder arm 22) may also be operably associated with the system 10, and, in particular, the mechanized system 51, to be actuated to actuate one or more operable portions of the tool module 14. The distal end of the transmission member 30 may be operably associated with the operable portion of the tool module 14 to be actuated by the transmission member 30. A portion of the transmission member 30 proximal to the distal end of the transmission member 30 (such as, but not limited to the proximal end of the transmission member 30) is operably coupled with the robotic system 10, such as to the mechanized system 51, such as to the instrument 12. In some aspects, the mechanized system 51, such as the instrument 12, includes an actuator operably coupled with the transmission member 30 to transmit actuation forces from the system 10 to the transmission member 30 to actuate one or more components of the tool module 14. The transmission member 30 may be removable from the system 10 with the tool module 14. For instance, when the tool module 14 is disconnected from the robotic system 10, such as from the instrument 12, the transmission member 30 may be removed with the tool module 14. In some aspects, the transmission member 30 is separable from the instrument 12 at a location adjacent the end cap 20 (typically, although not necessarily, with a distalmost portion of the transmission member 30 remaining coupled with the tool module 14). In some aspects, the transmission member 30 is separable from the instrument 12 at a location proximal to the end cap 20. In some aspects, a component of the transmission member 30 remains with the system 10 after removal of the tool module 14 from the system 10. It will be appreciated that the location at which the transmission member 30 is coupled with the system 10 (e.g., the instrument 12) is not critical to the present disclosure. As such, the present disclosure is not limited by specific configurations of the connections of the transmission member 30 with the system 10 or by specific configurations of the structures and manners by which the transmission member 30 is disconnected from the system 10.

Referring to FIGS. 4 and 5, in a first illustrated example of an embodiment of a mounting system 100, a surgical tool module 114 is mechanically mounted to the distal end 16 of the insertion tube 18 via a bayonet fitting. The distal end 16 of the insertion tube 18 includes one or more radially extending pins 150 on a reduced diameter portion 152 of the insertion tube 18. The tool module 114 includes a proximal collar 154 having one or more L-shaped or J-shaped internal grooves 156 opening at a proximal end of the collar 154. The collar 154 of the tool module 114 can be received over the distal end 16 of the insertion tube 18, with the pins 150 guided into respective grooves (not shown) on the collar 154. Once the collar 154 is longitudinally advanced on the insertion tube 18, the shape of the grooves 156 permits the collar 154 to be rotated relative to the distal end 16 of the insertion tube 18, and the pins 150 to be captured by the shape of the grooves 156 to longitudinally lock the tool module 114 relative to the insertion tube 18. The outer diameter of the distal end 16 of the insertion tube 18 and the inner diameter of the collar 154 may be sized to inhibit inadvertent counter-rotation which would permit unintentional separation. Referring to FIG. 5, alternatively or additionally, where the grooves 156 are J-shaped and the tool module 114 is fully rotated on the pins 150, the inside of the collar 154 may include a spring-biased element 158 that forces the collar 154 to be positioned against the pins 150, with the pins 150 residing in a section of the grooves 156 to prevent rotation of the tool module 114 relative to the insertion tube 18. The tool module 14 may remain in his configuration until the spring force is manually or actively countered. The collar 154 is movable against the spring bias and rotated to release the bayonet connection.

Referring to FIG. 6, a second example of an embodiment of a mounting system 200 is another bayonet fitting in which the pins 250 are provided on a smaller diameter portion 252 of a tool module 214, and the grooves 256 are defined in a collar 254 at the distal end of 16 the insertion tube 18. The connection between the two components is otherwise as described with respect to mounting system 200.

Turning now to FIG. 7, a third example of an embodiment of a mounting system 300 for a tool module 314 is shown. The tool module 314 includes a threaded male end 350, and the distal end 16 of the insertion tube 18 is provided with an internally threaded rotating collar 354. The collar 354 can be rotated in a first direction about the threaded male end 350 to capture the tool module 314. The tool module 314 can be released by counter rotating the collar 354 in a second direction to loosen and release the threaded male end 350 of the tool module 314.

Referring now to FIGS. 8, 9, and 10, a fourth example of an embodiment of a mounting system 400 for a surgical tool module 414 is shown. The surgical tool module 414 includes a protruding generally cylindrical proximal end 450. The proximal end 450 can be smooth or textured. The distal end 16 of the insertion tube 18 is provided with a split shaft collet 452 with outer threads 454, and a nut 464 rotatable over the threads 454 of the collet 452. The proximal end 450 of the module 414 is inserted into the collet 452 when the collet 452 is in an open configuration (when the nut 464 is at a proximally displaced location along the outer threads 454). The nut 464 is then advanced onto the threads 454 to reduce the diameter of the collet 452 and tighten the collet 452 about the proximal end 450 of the tool module 414 to secure the tool module 414 to the insertion tube 18. The tool module 414 may be released by loosening the nut 464.

Turning now to FIG. 11, a fifth example of an embodiment of a mounting system 500 for a tool module 514 is shown. The surgical tool module 514 is secured to the distal end 16 of the insertion tube 18 via a set screw 550. The set screw 550 is received through an end hole 552 of the end cap 520 of the tool module 514 and into a set screw hole 554 in the end face 556 of the insertion tube 18. The set screw 550 locks and unlocks the tool module 514 relative to the insertion tube 18. Alternatively, the set screw 550 can be advanced through a hole in a side of the tool module 514 and into a corresponding hole in the side of the insertion tube 18.

Referring to FIG. 12, a sixth example of an embodiment of a mounting system 500a for a tool module 514a is shown. The tool module 514a includes a grub (set) screw 550a that is received through a hole 560a aligned with a screw boss 554a in the end cap 520a. The grub screw 550a locks and unlocks the tool module 514a relative to the insertion tube 18.

Turning now to FIGS. 13, 14 and 15, a seventh example of an embodiment of a mounting system 700 for a tool module 714 is shown. The end cap 720 of the tool module 714 defines a split ring having a side wall 770 and a lower lip 772 (e.g., a radially-inwardly extending lower lip 772). Two holes 774 are located on opposite sides of the split 776 in the end cap 720. A tubular element 750 with an external groove 752 is provided at the distal end 16 of the insertion tube 18. A tool, such as a pliers, can be placed into the two holes 774 and operated to expand the ring of the end cap 720 on either side of the split 776 (e.g., similarly to operation of a circlip) to position and secure the end cap 720 over the tubular element 750, with the lip 772 captured within the groove 752. While the end cap 720 is adapted to be self-retained on the tubular clement 750, a U-shaped retainer (not shown) can be placed within the two holes 774 to further secure the ends of the end cap 720 with respect to each other during the surgical procedure. After the procedure, the retainer can be removed and the tool is used to expand the end cap 720 and remove the tool module 714 from the insertion tube 18. A trapeze shaped retainer (not shown) can be permanently connected to the mounting holes. When pushed down, the mounting holes are slightly approximated to secure the end cap 720 with respect to the tubular element 750. After the procedure, to remove the end cap 720 from the endoscope, the retainer may be pushed away/upwards from the endoscope, which increases the distance between the mounting holes 774 and releases the end cap 720.

Referring now to FIGS. 16, 17, and 18, an eighth example of an embodiment of a mounting system 800 for a tool module 814 is shown. The tool module 814 includes a proximal bearing surface 850 defining one or more radially outwardly facing resilient retainers 852. The distal end 16 of the insertion tube 18 is provided with a rigid tubular wall portion 854 with one or more windows or recesses 856 sized and positioned to receive the retainers 852. The tubular wall portion 854 may be a metal tube, and the windows 856 may be laser cut into the wall of the metal tube 854. In assembly, the proximal bearing surface 850 is inserted into the tubular wall portion 854 until the retainers 852 enter the recesses 856 and result in capture of the module 814 at the distal end 16 of the insertion tube 18. The tool module 814 may be released by displacing the retainers 852 radially inwardly.

Referring to FIGS. 19, 20, and 21, a ninth example of an embodiment of a mounting system 900 for a tool module 914 is shown. The tool module 914 includes one or more inwardly facing resilient retainers 952. The distal end 16 of the insertion tube 18 includes a collar 954 with a circumferential groove 956 defining a radially-outwardly extending upper lip 958. The module 914 can be seated on the collar 954 such that the resilient retainers 952 are forced over the lip 958 and seat in the groove 956, with the retainers 952 retained by the lip 958. To further prevent rotation, the collar 954 may include spaced apart openings, longitudinal rails, and/or other guides along the collar 954 that permit the module 914 to mount only in a defined orientation relative to the distal end 16 of the insertion tube 18. The tool module 914 may be removed from the collar 954 by releasing the resilient retainers 952, which may require a specialized tool.

Referring to FIGS. 22, 23, and 24, a tenth example of an embodiment of a mounting system 1000 for a tool module 1014 is shown. The tool module 1014 includes a relatively short hook 1050 and one or more relatively longer snap retention arms 1052. The distal end 16 of the insertion tube 18 is provided with a collar 1054 defining first and second longitudinally spaced apart annular grooves 1056, 1058. The hook 1050 is adapted to be initially engaged in the first (distal) groove 1056 (FIG. 23). Then the end cap 1020 of the tool module 1014 is rotated (in the direction of arrow 1060) onto the distal end 16 of the insertion tube 18 until the retention arms 1052 engage in the second (proximal) groove 1058 (FIGS. 23 and 24). The tool module 1014 can be released with a tool that loosens the retention arms 1052 from the second groove 1058.

Turning now to FIG. 25, an eleventh example of an embodiment of a mounting system 1100 for a tool module 1114 is shown. The tool module 1114 includes a proximal retainer 1150. By way of example, the retainer 1150 can be provided in the form of a lip element extending (e.g., radially-outwardly) at least partially about an end portion of a tubular member 1142 extending proximally from the end cap 1120. The distal end 16 of the insertion tube 18 includes a window 1152 in its sidewall 1154. A clip 1156 is provided for insertion into the window 1152 and engagement with the retainer 1150. The clip 1156 has resilient arms 1160 insertable through the window to engage the retainer 1150. In some aspects, the window 1152 is shaped to correspond to the shape of the resilient arms 1160, such as wishbone-shaped as illustrated in FIG. 25. The resilient arms 1160 may have grips 1162 configured to engage about the retainer 1150. The clip 1156 may include a closure portion 1164 which engages the insertion tube to retain the clip 1156 with respect to the insertion tube 18. In some aspects, the closure portion 1164 has a radius of curvature matching the outside of the insertion tube 18. In some aspects, the closure portion 1164 includes recesses 1166 configured to facilitate engagement by a tool for removal of the clip 1146. When the proximal end of the module 1114 is inserted into the distal end 16 of the insertion tube, the clip 1156 is inserted into the window 1152 and retains the lip element 1150 from moving axially in a distal direction. This prevents release of the tool module 1114 from the insertion tube 18. The clip 1156 can be removed via engagement of, e.g., a tool, with the recesses 1166. This then permits release of the tool module 1114 from the insertion tube 18.

Referring now to FIGS. 26 and 27, a twelfth example of an embodiment of a mounting system 1200 for a tool module 1214 is shown. The tool module 1214 includes an end cap 1220 with one or more proximally extending forks 1252, 1254 that each define partial portions 1256, 1258 of an annular groove. The distal end 16 of the insertion tube 18 includes one or more longitudinal alleys (corresponding to the one or more forks 1252, 1254), with one alley 1260 shown and another hidden on the opposite side of the insertion tube 18. The forks 1252, 1254 can be received in the alleys 1260. In the illustrated example of an embodiment, the alleys 1260 are provided on an exterior surface of the insertion tube 18. However, other configurations (e.g., cut-outs) are within the scope of the present disclosure as may be appreciated by one of ordinary skill in the art. The distal end 16 of the insertion tube 18 also defines the remaining portions 1262, 1264 of the annular groove 1266. The groove 1256 may be formed as a recess or by a space between two longitudinally displaced walls. When the forks 1252, 1254 are fully inserted into the alleys 1260, the end cap 1220 seats against the distal end 16 of the insertion tube 18 and the complete annular groove 1266 is formed. A band, tie wrap, adhesive tape, clamp, or other, e.g., band-like, element 1268 configured and shaped to fit over and/or in the annular groove 1266 is provided within the annular groove 1266 to lock the tool module 1214 relative to the insertion tube 18. After the procedure, the element 1268 can be cut, or opened, or otherwise removed, and the tool module 1214 can be removed from the insertion tube 18.

Turning now to FIG. 28, a thirteenth example of an embodiment of a mounting system 1300 for a tool module 1314 is shown. The tool module 1314 includes a proximal skirt 1350 defining gripping ridges 1352 and an alignment slot 1354. The distal end 16 of the insertion tube 18 includes a distal skirt 1360 with one or more tabs 1362, such as one or more laser cut barb-like tabs 1362, adapted to bear against the gripping ridges 1352 and one or more longitudinal deformations in the distal skirt 1360 structured as a rib 1364. The tool module 1314 is rotationally oriented relative to the insertion tube 18 by guiding the rib 1364 into the slot 1354. Then, the gripping ridges 1352 may be forced past the ends of the tabs 1362 until the tool module 1314 is fully seated with respect to the insertion tube 18. Interference between the ridges 1352 and the tabs 1362 prevents inadvertent release of the tool module 1314. The tool module 1314 can subsequently be released with a tool that outwardly deforms the tabs 1362.

Referring to FIG. 29, a fourteenth example of an embodiment of a mounting system 1300a, substantially similar to mounting system 1300, shows an opposite/reverse arrangement in which the proximal skirt 1350a on the tool module 1314a includes the tabs 1362a and alignment ridges (not shown), and the distal skirt 1360a on the insertion tube 18 includes the gripping ridges 1352a and alignment slot 13654a. The components can be similarly assembled and disassembled.

Turning now to FIG. 30, a fifteenth example of an embodiment of a mounting system 1400 for a surgical tool 1414 module is shown. The tool module 1414 includes a proximal tubular portion 1450, for example in the form of skirt 1450, with a first through hole 1452. The distal end 16 of the insertion tube 18 includes a corresponding second through hole 1454. When the skirt 1450 of the tool module 1414 is fully inserted into the distal end 16 of the insertion tube 18, the first and second through holes 1452, 1454 align. A split pin 1456 is advanced through the aligned first and second holes 1452, 1454 to secure the tool module 1414 relative to the insertion tube 18. The pin 1456 can be removed to permit removal of the tool module 1414 relative to the insertion tube 18.

Referring now to FIG. 31, a sixteenth example of an embodiment of a mounting system 1500 for a tool module 1514 is shown. The tool module 1514 includes a proximally extending key member 1550, while the distal end 16 of the insertion tube 18 includes a recessed plate 1552 defining a keyway 1554. The key member 1550 of the tool module 1514 is positioned into the plate 1552 and rotated to secure the tool module 1514 with respect to the insertion tube 18. A tool (not shown) may be used to rotate the key member 1550. The tool module 1514 may then be released by rotating the key member 1550 to align the key member 1550 with the keyway 1554.

Turning now to FIG. 32, a seventeenth example of an embodiment of a mounting system 1600 for a tool module 1614 is shown. The tool module 1614 is provided with a wire tether 1650 that extends from a base 1652 of the end cap 1620, through the insertion tube 18 to a point of securement (not shown). The distal end of the tether 1650 may include a nipple 1654 that is pulled in tension against the base 1652 of the end cap 1620. Alternatively, the distal end of the tether 1650 may be a loop secured through a hole in the end cap 1620. The proximal end of the tether 1650 may extend to a location adjacent an actuating end of the insertion tube 18. Alternatively, the proximal end of the tether 1650 may be secured at an intermediate location along the length of the insertion tube 18. After the procedure, the tool module 1614 can be released by releasing or cutting the tether 1650.

Referring to FIGS. 33, 34, and 35, an eighteenth example of an embodiment of a mounting system 1700 for a tool module 1714 is shown. The mounting system 1700 includes a pliable over-cap 1750 with a circumferential portion 1752, an upper shoulder 1754, and a tab 1756. The distal end 16 of the insertion tube 18 is provided with a collar 1760 having an annular groove 1762 defining a distal lip 1764 (e.g., a radially-outwardly extending distal lip 1764). The tool module 1714 is assembled at the distal end of the collar 1760. The over-cap 1750 is positioned onto the tool module 1714 such that the shoulder 1754 seats against an upper portion 1766 of the end cap 1720 of the tool module 1714. Then, a crimping tool (not shown) is used to crimp the circumferential portion 1752 of the over-cap 1750 into the annular groove 1762 of the collar 1760 to retain the tool module 1714 relative to the insertion tube 18 by capture of the end cap 1720 of the tool module 1714 between the upper shoulder 1754 and the crimped circumferential portion 1752 of the over-cap 1750. When it is intended to remove the tool module 1714 from the distal end 16 of the insertion tube 18, the tab 1756 may be peeled back to release and remove the over-cap 1750. In some aspects, the tab 1756 is frangible and/or at least a portion of the over-cap 1750 may be split so that the tool module 1714 can be released from the insertion tube 18. A tool (not shown) may be used to aid in pulling the tab 1756.

Turning now to FIGS. 36, 37, and 38, a nineteenth example of an embodiment of a mounting system 1800 for a tool module 1814 is shown. The tool module 1814 is positionable at the distal end 16 of the insertion tube 18. The mounting system 1800 includes a collar 1850 at the distal end 16 of the insertion tube 18. In the example of an embodiment illustrated in FIGS. 36, 37, and 38, the collar 1850 includes one or more lugs 1852 (e.g., two diametrically opposed lugs 1852). A strap 1854, such as an elastic strap 1854, extends across the lugs 1852 and retains the tool module 1814 with respect to the distal end 16 of the insertion tube 18. The tool module 1814 can be removed by releasing at least one end of the elastic strap 1854 from one of the lugs 1852.

Referring now to FIG. 39, a twentieth example of an embodiment of a mounting system 1900 for a tool module 1914 is shown. The mounting system 1900 includes a magnet 1952 provided with respect to (e.g., mounted on or otherwise coupled to) at least one of the insertion tube 18 and the surgical module 1914. In the illustrated example of an embodiment, the mounting system 1900 includes a magnet 1952 at a distal end 16 of the insertion tube 18. The magnet 1952 may be provided internal to the insertion tube 18 and may be a portion of an alignment feature to rotationally align the tool module 1914 on the distal end 16 of the insertion tube 18. In some aspects, the magnet 1952 is provided as a part of a tubular member 1944 at a distal end of a tool channel defined in the insertion tube 18. Such tubular member 1944 is adapted to attract the tubular guide 1942 at the proximal end of the tool module 1914. Alternatively, other components of the tool module 1914 and the insertion tube 18 can have sufficient magnetic attraction with respect to one another to retain the tool module 1914 in place with respect to the insertion tube 18 and against inadvertent release therefrom. In such cases, the magnetic attraction is sufficiently strong to retain the tool module 1914 against inadvertent removal but permits active removal by pulling counter to the magnetic force with a force greater than forces encountered during a surgical procedure. As yet another alternative, the insertion tube 18 can be provided with an electromagnetic system 1970 which, when activated, attracts the tool module 1914, and, when deactivated, releases the tool module 1914 relative to the insertion tube 18.

Turning now to FIGS. 40 and 41, a twenty-first example of an embodiment of a mounting system 2000 for a tool module 2014 is shown. The tool module 2014 is fitted with respect to the distal end 16 of the insertion tube 18, such as with a press fit. The distal end 16 of the insertion tube 18 may include a reduced diameter collar 2050 to facilitate the fit. Then, a securing element 2054 is provided over the assembly. In some aspects, the securing element includes a tape 2054 or other structure configured to be secured over the insertion tube 18 and the tool module 2014.

Referring to FIGS. 42 and 43, in another mounting configuration, the securing element includes a heat shrink tube 2054a provided about the interface of a tool module 2014 and the insertion tube 18, and then heated to shrink and provide a secure joint. The tool module 2014 and the insertion tube 18 can be separated by removing the tape 2054 or heat shrink tubing 2054a and applying sufficient force to separate the components.

Turning now to FIGS. 44 and 45, a twenty-second example of an embodiment of a mounting system 2100 for a tool module 2114 is shown. In the mounting system 2100, a proximal end of the tool module 2114 is formed as a male connector 2150. The male connector 2150 has one or more exterior grooves 2152, and, optionally, one or more o-rings 2154 installed in each of the grooves 2152. The male connector 2150 is inserted into a female connector 2160 formed at the distal end 16 of the insertion tube 18. The o-rings 2154 provide a secure temporary assembly between the two connectors 2150, 2160.

Alternatively, as shown in FIGS. 46 and 47, the distal end 16 of the insertion tube 18 can be provided with a male connector 2150a which is provided with grooves 2152a and, optionally, o-rings 2154a; and the tool module 2114a may be provided with a female connector 2160a into which the males connector 2150a is inserted to form a secure temporary assembly 2100a.

Referring now to FIGS. 48 and 49, a twenty-third example of an embodiment of a mounting system 2200 for a tool module 2214 is shown. The mounting system 2200 includes an adhesive 2280 applied between a portion of the tool module 2214 and a portion of the insertion tube 18 or a portion connected to the insertion tube 18. The tool module 2214 may include a skirt 2250 and/or the insertion tube 18 may include a collar 2260 to provide increased surface area with respect to which the adhesive 2280 may be applied and/or may act. The adhesive 2280 may be, for example, a quick-setting adhesive such as cyanoacrylate, a quick set epoxy, or an adhesive with activator to accelerate curing. The adhesive 2280 may be, for example, a UV curable adhesive such as Loctite® light cure adhesive or Dymax® UV epoxies. The tool module 2214 can be released from the distal end 16 of the insertion tube 18 using a deactivator for the adhesive, mechanical release, and/or steam cleaning.

Turning now to FIGS. 50 and 51, a twenty-fourth example of an embodiment of a mounting system 2300 for a tool module 2314 is shown. The mounting system 2300 includes a coil spring 2350 attached to and extending from a proximal end of the tool module 2314. The coil spring 2350 naturally has an inner diameter smaller than the outer diameter of the insertion tube 18 (or at least a distal portion 16 thereof). However, the coil spring 2350 can be compressed in length to enlarge its inner diameter to a dimension greater than the outer diameter of the distal portion 16 of the insertion tube 18. In this compressed configuration, the distal portion 16 of the insertion tube 18 is advanced into contact with the tool module 2314. Then, the coil spring 2350 is released, and is naturally biased to return to its pre-compressed length and diameter, thereby gripping the outer surface of the insertion tube 18 as the inner diameter of the coil spring 2350 automatically is reduced. The components are retained by compression and friction. The components can be released by again compressing the length of the coil spring 2350. Tools (not shown) may be provided for compressing the coil spring 2350 whether on or off of the insertion tube 18.

Referring to FIGS. 52 and 53, a twenty-fifth example of an embodiment of a mounting system 2400 for a tool module 2414 is shown. The mounting system 2400 includes an elastomer roll sock component 2450 attached to the tool module 2414. The tool module 2414 is arranged at the distal end 16 of the insertion tube 18, and the sock 2450 is unrolled over the distal end 16 of the insertion tube 18 to hold the tool module 2414 in position by way of compression and friction. After a procedure, the tool module 2414 can be released by re-rolling the sock 2450, or tearing or cutting the sock 2450 away from the insertion tube 18.

Referring now to FIGS. 54 and 55, a twenty-sixth example of an embodiment of a mounting system 2500 for a tool module 2514 is shown. The mounting system 2500 includes a pliable crimp band 2550 formed at a proximal portion of the tool module 2514. For example, the crimp band 2550 may extend from the end cap 2520. The crimp band 2550 may include one or more deformable arms 2552 adapted to be deformed to extend about and overlap about the circumference of distal end 16 of the insertion tube 18. The ends of the arms 2552 may define interfering shapes, such as a recess 2554 and an arrow 2556 that fits within the recess 2554 to prevent unintended release of the arms 2552 once deformed into a desired position. The tool module 2514 is coupled at the distal end 16 of the insertion tube 18, and the arms 2552 of the crimp band 2550 are deformed about the distal end 16 of the insertion tube 18. A sacrificial sleeve (not shown) may be placed onto the insertion tube 18 under or over the crimp band 2550 to protect the insertion tube 18, patient, and/or other tools from any damage by the crimp band 2550. A tool (not shown) may be provided for assembly and disassembly of the mounting system 2500.

It will be appreciated that in any of the above-described embodiments, and in any further embodiments in accordance with various principles of the present disclosure, the distal end 16 of the insertion tube 18 may itself be modified, or a separate element with the various structures and features for coupling with respect to the tool module may be operably coupled to the insertion tube 18. Moreover, it will be appreciated that although the above-described embodiments are particularly suitable for use with and operable association with a robotic instrument of a robotic system, principles of the present disclosure are applicable to manually-operated (e.g., traditional human-controlled, and not predominantly computer-controlled) devices and systems as well.

In any embodiment in which a sharp may be exposed at or adjacent the temporary coupling of the tool module and the distal end of the insertion tube, the temporary joint of such components may be covered by a removable sleeve or tape or otherwise to prevent injury or damage to the patient or other instruments. Similarly, in any of the embodiments having a potentially inadvertently releasable element, the element can be secured with tape or a sleeve or otherwise to secure the components together. By way of example only, the joint, components, and proximate areas may be covered in a polyether block amide polymer (e.g., Pebax®) tube, heat shrink tube, adhesive tape, or other material to cover the joint, hold components in position, and/or maintain a smooth liquid-tight interface between the components.

There have been described and illustrated herein embodiments of tool modules, such as robotic suturing systems for bariatric treatments, and methods of coupling devices and systems with robotic systems. While particular embodiments have been described, it is not intended that the present disclosure be limited to those specific embodiments, as it is intended that the present disclosure be as broad in scope as the art will allow and that the specification be read likewise. It will therefore be appreciated by those of ordinary skill in the art that various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. For example, various features of the disclosure are grouped together in one or more aspects, embodiments, or configurations for the purpose of streamlining the disclosure. However, it should be understood that various features of the certain aspects, embodiments, or configurations of the disclosure may be combined in alternate aspects, embodiments, or configurations. While the disclosure is presented in terms of embodiments, it should be appreciated that the various separate features of the present subject matter need not all be present in order to achieve at least some of the desired characteristics and/or benefits of the present subject matter or such individual features. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open- ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A system for performing a procedure with respect to a patient, said system comprising:
a robotic instrument operably coupled with a robotic system for operative control of said robotic instrument, and having and elongate member configured for insertion through a natural orifice of the patient; and
a tool module operably couplable with said elongate member of said robotic instrument, and configured to perform a procedure on a patient;
wherein said robotic instrument is operably coupled with the robotic system for control of movement of said robotic instrument and said tool module.

2. The system of claim 1, wherein said tool module is coupled to the distal end of said elongate member via a mechanical connection.

3. The system of claim 2, wherein said mechanical connection comprises interengaging elements on said elongate member and said tool module.

4. The system of claim 3, wherein said mechanical connection is selected from the group consisting of: a bayonet lock; a threaded connection; a collet on one of said elongate member and said tool module and a compressible portion on the other of said elongate member and said tool module, the collet adapted to compress the compressible portion; a set screw configured to secure said tool module relative to said elongate member; a split ring; at least one resilient retainer; at least one hook and at least one resilient retention arm; at least one clip; a banded element; at least deformable tab and at least one ridge against which the at least one deformable tab interferes; a pin insertable into a hole extending through the distal end and the tool module; or a key and a keyway.

5. The surgical system of claim 1, wherein said tool module is coupled to said elongate member by a separate structure positioned over said tool module and said elongate member to retain said tool module with respect to said elongate member.

6. The system of claim 5, wherein said separate structure is selected from the group consisting of: a crimpable collar; an elastic retaining strap; a heat shrinkable element; at least one o-ring; a resilient diametrically reducible member; or an unrollable sock.

7. The system of claim 1, wherein said tool module is coupled to said elongate member via a magnetic coupling.

8. The system of claim 1, wherein said tool module is coupled to said elongate member via an electromagnetic coupling.

9. The system of claim 1, wherein said tool module is coupled to said elongate member via an adhesive coupling.

10. The system of claim 1, further comprising a mechanized system operably associated with said elongate member and the robotic system for input therefrom to control movement of said elongate member and/or said tool module based on input from the robotic system.

11. The system of claim 10, further including sensors operably associated with said robotic instrument and said mechanized system and usable to control operation of said robotic instrument.

12. A system for performing a procedure with respect to a patient, said system comprising:
an instrument comprising an elongate member having a distal end;
a tool module configured to perform a procedure with respect to a patient; and
a mounting system comprising at least one of a mechanical connection, a magnetic and/or
electromagnetic connection, a friction fit connection, an adhesive connection, or a separate element provided over a portion of said distal end of said elongate member and a portion of said tool module to retain said tool module operably coupled with respect to said elongate member and configured to transmit movements from said instrument to said tool module to operate said tool module to perform the procedure with respect to the patient.

13. The system of claim 12, wherein said tool module comprises a suturing tool.

14. The system of claim 13, wherein said suturing tool comprises a fastener holder arm adapted to rotate a releasable tissue fastener through a path.

15. The system of claim 14, wherein said releasable tissue fastener is a suture needle coupled with a length of suture.

16. A method of performing a procedure with respect to a patient using a robotic system, said method comprising:
operably coupling a first tool module with respect to an elongate member of a medical instrument via a mounting system;
operating the medical instrument via the robotic system to operate the first tool module to perform a procedure with respect to the patient; and
removing the first tool module from the elongate member.

17. The method of claim 16, further comprising sensing the position and/or shape of the elongate member with at least one sensor operably associated with the elongate member, and using the robotic system to utilize signals generated by the at least one sensor to control movement of the elongate member and/or the first tool module.

18. The method of claim 16, further comprising operably coupling a second tool module with respect to the elongate member and operating the medical instrument via the robotic system to operate the second tool module to perform a different procedure with respect to the patient than performed by the first tool module.

19. The method of claim 16, further comprising utilizing a mechanized system operably associated with the robotic system and the medical instrument to control at least a component of the medical instrument and/or the tool module.

20. The method of claim 16, wherein the procedure is a suturing procedure.
